(19) United States
(12) Patent Application Publication
(10) Pub. No.: US 2021/0251534 A1
(43) Pub. Date: Aug. 19, 2021

(54) System and Method for Determining a Current Hemoglobin Level

(71) Applicant: Mayo Foundation for Medical Education and Research, Rochester, MN (US)

(72) Inventors: Kalyan S. Pasupathy, Rochester, MN (US); Devashish Das, Rochester, MN (US); Mustafa Y. Sir, Rochester, MN (US); Martin D. Zielinski, Rochester, MN (US); Susan Hallbeck, Rochester, MN (US)

(21) Appl. No.: 17/251,348

(22) PCT Filed: Jun. 26, 2019
(86) PCT No.: PCT/US2019/039345

§ 371 Date: Dec. 11, 2020

Related U.S. Application Data
(60) Provisional application No. 62/690,159, filed on Jun. 26, 2018.

(51) Int. Cl. A61B 5/145; G16H 40/63; G16H 50/50; G16H 50/70

(52) U.S. Cl. CPC A61B 5/14546; G16H 50/70; G16H 50/50; G16H 40/63

(57) ABSTRACT

An apparatus and computerized method of determining a current hemoglobin level comprising providing a computing device having an input/output interface, one or more processors and a memory, receiving a series of hemoglobin measurements taken within a time window, determining a current hemoglobin level based on the series of hemoglobin measurements using a linear regression model, and providing the current hemoglobin level via the input/output interface.

### CROSS-REFERENCE TO RELATED APPLICATIONS

None.

### TECHNICAL FIELD OF THE INVENTION

The present invention relates in general to medical systems, and more particularly to a system and method for determining a current hemoglobin level.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with blood loss, hemogoblin levels and transfusions.

Hemorrhagic shock is a life threatening medical condition caused by severe blood loss. It is the most common cause of preventable death in injured patients, the majority of which is due to uncontrolled hemorrhage [2]. Blood transfusion decisions in current medical practice are limited to vital signs until laboratory guided hemoglobin (HgB) values are available [3]. Unfortunately, due to the inherent delays of these laboratory based tests, the patient's physiologic status is no longer represented by this delayed lab value. As a result, insufficient or unnecessary blood transfusions occur [4] and therefore an innovative solution is required to optimize blood transfusions.

SpHb (Radical-7 Pulse CO-Oximeter; Masimo, Inc., Irvine, Calif.) is an FDA-approved, real-time, non-invasive technology, which utilizes seven different light wavelengths to measure HgB values at every 2-second interval. Knowing the real-time HgB values has great potential to accurately guide blood transfusions in hemorrhaging trauma patients. There is, however, discrepancy within the current literature as to device accuracy. For instance, while Frasca et al. [5] demonstrated correlation to gold standard laboratory-based HgB measurements with a bias and 95% limits of agreement measured at 0.0±1.0 g/dL on the Bland Altman plot in 474 samples from 62 critically ill, non-hemorrhaging patients, the SpHb had poor correlation in a separate study of 23 hemorrhaging trauma patients showing a Spearman R² of 0.47 (R=0.47=0.69) in Joseph et al. [6]. The significant potential of this device to guide decisions for transfusion in trauma care entails further research to improve its accuracy. It appears that systematic studies aimed at improving the accuracy of SpHb monitors have not been reported in the literature. Besides, factors affecting the accuracy of SpHb also have not been conclusively established [7], making it difficult to account for all these factors.

### SUMMARY OF THE INVENTION

The SpHb measurement error, defined as the difference between SpHb and gold standard HgB measurements taken concurrently, is correlated with the magnitude of the true HgB levels. Various embodiments of the present invention accurately estimate true levels of hemoglobin through a transformation of prior SpHb measurements thus improving the accuracy of the current SpHb measurements.

A system and method of determining hemoglobin levels that improves the accuracy of SpHb monitors is described herein. SpHb monitors are non-invasive hemoglobin monitoring tools with the potential to improve critical care protocols in trauma care. In one embodiment, the system and method are based on fitting smooth spline functions to SpHb measurements collected over a time window and then using a functional regression model to predict the true HgB value for the end of the time window. In such an embodiment, the accuracy of the system and method described herein provided a reduced mean absolute error of 1.08 g/Dl [1] as compared to the mean absolute error between the raw SpHb measurements and the gold standard hemoglobin measurements of 1.26 g/Dl. As described herein, true levels of hemoglobin can be accurately estimated through appropriate transformation of prior SpHb measurements to improve the accuracy of the current SpHb measurement.

One embodiment of the present invention provides a computerized method of determining a current hemoglobin level comprising: providing a computing device having an input/output interface, one or more processors and a memory; receiving a series of hemoglobin measurements taken within a time window; determining a current hemoglobin level based on the series of hemoglobin measurements using a linear regression model; and providing the current hemoglobin level via the input/output interface. In one aspect, the method further comprises selecting the time window based on a specified number of hemoglobin measurements. In another aspect, the determining and providing steps are performed only after the time window is complete. In another aspect, the time window is a rolling time window and the determining and providing steps are repeated for each new hemoglobin measurement. In another aspect, the method further comprises selecting the linear regression model from a group of at least two models comprises: yᵢ=a₀x_{i,N}_{ᵢ}+b where yᵢ is the current hemoglobin level, a is a first coefficient, x_{i,N}_{ᵢ} is the hemoglobin measurement, and b is a second coefficient; yᵢ=Σⱼ₌₀^{M}aⱼx_{i,N}_{ᵢ}₋ⱼ+b where M is a number of hemoglobin measurements taken within the time window; yᵢ=β₀+∫₀²A(t)xᵢ(t)dt where β₀ is an intercept term, xᵢ(t) is a smoothing function, and A(t) is a smooth coefficient function that establishes a relationship between yᵢ and xᵢ(t); or yᵢ=b+a\hat{ξ}_{i,p} where \hat{ξ}_{i,p} are principal component scores that quantify the variability of xᵢ(t) along ψₚ(t), and ψₚ(t) are functional principal components of xᵢ(t) explaining the variability in xᵢ(t). In another aspect, the method further comprises receiving or determining the first coefficient and the second coefficient. In another aspect, the method further comprises smoothing the series of hemoglobin measurements using a B-spline basis function. In another aspect, the series of hemoglobin measurements are received from the input/output interface, or one or more sensors communicably coupled to the one or more processors, or the memory. In another aspect, a time interval between the hemoglobin measurements is not equally spaced. In another aspect, the series of hemoglobin measurements includes missing data or outlier data. In another aspect, the method further comprises adjusting the current hemoglobin level based on one or more prior laboratory determined hemoglobin levels. In another aspect, the method further comprises sending an alert via the input/output interface whenever the current hemoglobin level or a rate of change of the current hemoglobin level is outside one or more limits. In another aspect, the method further comprises receiving one or more patient factors via the input/output interface, one or more sensors or one or more modules. In another aspect, the one or more patient factors comprise a heart rate, an arterial oxygen saturation, an intravascular volume, a Pleth variability index, a perfusion index, a total oxygen content, an age, an injury type, a Glasgow coma index, a sex, a body mass index, a systolic blood pressure, a diastolic blood pressure, or a transfusion target range. In another aspect, the method further comprises adjusting the current hemoglobin level based on the one or more patient factors. In another aspect, the one or more sensors or the one or more modules comprise a pulse oximeter, an intravascular volume estimator, or a patient medical data source. In another aspect, the method further comprises: determining a blood product of fluid amount, a blood product of fluid type, a transfusion rate, or a transfusion timing based on the current hemoglobin level and the one or more patient factors; and providing the blood product or fluid amount, the blood product or fluid type, the transfusion rate, or the transfusion timing via the input/output interface. In another aspect, the method further comprises administering the blood product or fluid amount of blood product or fluid type to a patient at the transfusion rate and transfusion timing using one or more devices communicably coupled to the input/output interface. In another aspect, the input/output interface comprises a remote device, and the remote device is communicably coupled to the one or more processors via one or more networks. In another aspect, the computing device comprises a server computer, a workstation computer, a laptop computer, a mobile communications device, a personal data assistant, or a medical device.

Another embodiment of the present invention provides an apparatus for determining a current hemoglobin level comprising: an input/output interface; a memory; and one or more processors communicably coupled to the input/output interface and the memory, wherein the one or more processors receive a series of hemoglobin measurements taken within a time window, determine a current hemoglobin level based on the series of hemoglobin measurements using a linear regression model, and provide the current hemoglobin level via the input/output interface. In one aspect, the one or more processors receive a selection of the time window based on a specified number of hemoglobin measurements via the input/output interface. In another aspect, the one or more processors determine and provide the current hemoglobin level only after the time window is complete. In another aspect, the time window is a rolling time window and the one or more processors determine and provide the current hemoglobin level for each new hemoglobin measurement. In another aspect, the one or more processors receive a selection of the linear regression model from a group of at least two models comprising: yᵢ=a₀x_{i,N}_{ᵢ}+b where yᵢ is the current hemoglobin level, a is a first coefficient, x_{i,N}_{ᵢ} is the hemoglobin measurement, and b is a second coefficient; yᵢ=Σⱼ₌₀^{M}aⱼx_{i,N}_{ᵢ}₋ⱼ+b where M is a number of hemoglobin measurements taken within the time window; yᵢ=β₀+∫₀²A(t)xᵢ(t)dt where β₀ is an intercept term, xᵢ(t) is a smoothing function, and A(t) is a smooth coefficient function that establishes a relationship between yᵢ and xᵢ(t); or yᵢ=b+a\hat{ξ}_{i,p} where \hat{ξ}_{i,p} are principal component scores that quantify the variability of xᵢ(t) along ψₚ(t), and ψₚ(t) are functional principal components of xᵢ(t) explaining the variability in xᵢ(t). In another aspect, the one or more processors receive or determine the first coefficient and the second coefficient. In another aspect, the one or more processors smooth the series of hemoglobin measurements using a B-spline basis function. In another aspect, the series of hemoglobin measurements are received from the input/output interface, or one or more sensors communicably coupled to the one or more processors, or the memory. In another aspect, a time interval between the hemoglobin measurements is not equally spaced. In another aspect, the series of hemoglobin measurements includes missing data or outlier data. In another aspect, the one or more processors adjust the current hemoglobin level based on one or more prior laboratory determined hemoglobin levels. In another aspect, the one or more processors send an alert via the input/output interface whenever the current hemoglobin level or a rate of change of the current hemoglobin level is outside one or more limits. In another aspect, the one or more processors receive one or more patient factors via the input/output interface, one or more sensors or one or more modules. In another aspect, the one or more patient factors comprise a heart rate, an arterial oxygen saturation, an intravascular volume, a Pleth variability index, a perfusion index, a total oxygen content, an age, an injury type, a Glasgow coma index, a sex, a body mass index, a systolic blood pressure, or a diastolic blood pressure. In another aspect, the one or more processors adjust the current hemoglobin level based on the one or more patient factors. In another aspect, the one or more sensors or the one or more modules comprise a pulse oximeter, an intravascular volume estimator, or a patient medical data source. In another aspect, the one or more processors: determine a blood product or fluid amount, a blood product or fluid type, a transfusion rate, or a transfusion timing based on the current hemoglobin level and the one or more patient factors; and provide the blood product or fluid amount, the blood product of fluid type, the transfusion rate, or the transfusion timing via the input/output interface. In another aspect, the blood product or fluid amount of the blood product or fluid type are administered to a patient at the transfusion rate and the transfusion timing using one or more devices communicably coupled to the input/output interface. In another aspect, the input/output interface comprises a remote device, and the remote device is communicably coupled to the one or more processors via one or more networks. In another aspect, the apparatus comprises a server computer, a workstation computer, a laptop computer, a mobile communications device, a personal data assistant, or a medical device.

Moreover, the method can be implemented using a non-transitory computer readable medium that when executed causes the one or more processors to perform the method.

### BRIEF DESCRIPTION OF THE DRAWINGS

For a more complete understanding of the features and advantages of the present invention, reference is now made to the detailed description of the invention along with the accompanying figures and in which:

FIG. 1 is a scatter plot of SpHb measurements and corresponding gold standard Hgb measurements taken concurrently;

FIG. 2A is a plot of the correlation between SpHb measurement error calculated as yᵢ−x_{i,N}_{ᵢ} and BMI (−0.22) in accordance with one embodiment of the present invention;

FIG. 2B is a plot of the correlation between SpHb measurement error calculated as yᵢx_{i,N}_{ᵢ} and age (−0.07) in accordance with one embodiment of the present invention;

FIG. 3 is a graph in which the x-axis shows the actual predication error and the y-axis shows the value of the prediction error used to quantify the accuracy of the device, wherein any error less than 0.5 and greater than −0.5 is not considered in accordance with one embodiment of the present invention;

FIG. 4 is a plot of the smoothin SpHb measurements for patient 3 listed in Table 1 in accordance with one embodiment of the present invention;

FIG. 5 is a graph depicting the coefficient function in accordance with one embodiment of the present invention;

FIG. 6 is a plot of the smoothed SpHb measurements from 61 patients in accordance with one embodiment of the present invention;

FIG. 7A is a plot of the mean function of xᵢ(t)s in accordance with one embodiment of the present invention;

FIG. 7B is a plot of the principal component that explains 92% variability of xᵢ(t)s in accordance with one embodiment of the present invention;

FIGS. 8A-8D are histograms depicting the mean absolute error for different colincial conditions: pre-hospital transfusion (FIG. 8A), patient received blood transfusion in the hospital (FIG. 8B), anticoagulant or bleeding disorder (FIG. 8C), and type of trauma (FIG. 8D) in accordance with one embodiment of the present invention;

FIG. 9 is a block diagram of an apparatus in accordance with one embodiment of the present invention;

FIG. 10 is a flow chart of a method in accordance with one embodiment of the present invention;

FIGS. 11A-11F are images of various views of an apparatus in accordance with another embodiment of the present invention;

FIG. 12 is an image of a HGB picker widget screen in accordance with the embodiment of FIGS. 11A-11F;

FIG. 13 is an image of a details page screen in accordance with the embodiment of FIGS. 11A-11F; and

FIG. 14 is an image of a patient page screen in accordance with the embodiment of FIGS. 11A-11F.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The effectiveness of a simple linear transformation of SpHb measurements in improving estimation accuracy was tested and can be used in some embodiments. While a linear transformation seems to improve accuracy, in some cases, it may not be practical as the SpHb monitor needs to be disconnected from the patient from time to time and like most physiological signal measuring devices, the measurement can be noisy. These issues create certain difficulties, like missing data and outliers, in developing linear transformation models. Such issues are common in other longitudinal datasets [8] and functional data analysis methods have been used to address these issues [9]. Therefore, in one embodiment to address these issues, splines were used to smooth the noisy SpHb data and establish a functional regression model that uses the smoothed SpHb measurements as input and provides a modified estimate of the hemoglobin level. As tested, this approach was found to provide more accurate estimation of HgB levels.

The data used in during testing of the system and method consisted of 61 gold standard laboratory HgB measurements and corresponding SpHb measurements, which were recorded concurrently. For each of the 61 observations, SpHb measurements collected over a two hour period prior to the gold standard measurement was also available. The data were collected from 14 trauma patients who were being treated at a major academic medical center. Details about the medical condition of the patients, number of gold standard HgB measurements, as well as the age, sex, height, and weight of the patient, are given in Table I below.

TABLE 1
Detailed information about the patient data studied

| Patient | | | | | | | Sample |
|---|---|---|---|---|---|---|---|
| ID | Age | Sex | Weight | Height | BMI | Complaint | Size |
| 1 | 62 | F | 79.10 | 1.55 | 32.92 | Not Available | 3 |
| 2 | 87 | F | 62.00 | 1.62 | 23.62 | Fall | 1 |
| 3 | 87 | F | 94.50 | 1.68 | 33.48 | Car v Pedestrian | 7 |
| 4 | 60 | M | 138.00 | 2.03 | 33.49 | Gun shot | 5 |
| 5 | 62 | M | 78.00 | 1.86 | 22.55 | MVC | 6 |
| 6 | 69 | M | 59.30 | 1.68 | 21.01 | Not Available | 6 |
| 7 | 62 | M | 93.40 | 1.93 | 25.07 | Not Available | 4 |
| 8 | 23 | M | 65.20 | 1.85 | 19.05 | Fall out of | 1 |
| | | | | | | moving car | |
| 9 | 22 | M | 55.50 | 1.63 | 20.89 | MVC | 2 |
| 10 | 68 | M | 142.00 | 1.92 | 38.52 | Tree fell on | 3 |
| | | | | | | patient | |
| 11 | 72 | M | 77.40 | 1.73 | 25.86 | Abdominal | 5 |
| | | | | | | bleeding | |
| 12 | 77 | M | 73.60 | 1.67 | 26.39 | GI bleed | 12 |
| 13 | 19 | M | 88.00 | 1.78 | 27.77 | Tractor vs | 4 |
| | | | | | | automobile MVC | |
| 14 | 26 | M | 60.70 | 1.80 | 18.73 | Blunt abdominal | 2 |
| | | | | | | trauma | |

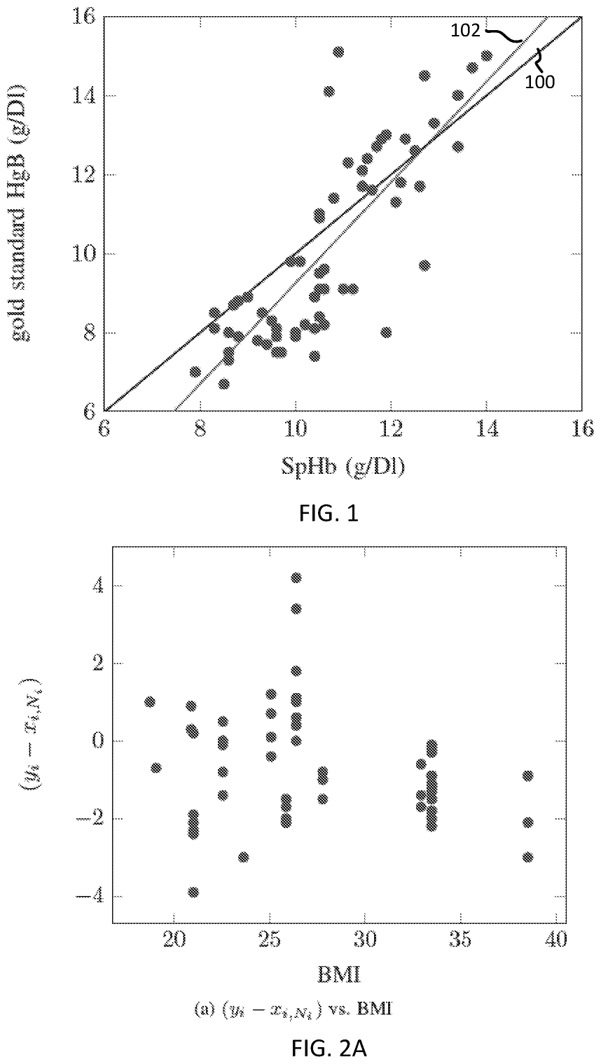

FIG. 1 shows the scatter plot of SpHb measurements and corresponding gold standard HgB measurements. The red solid line 102 is a regression line fit to these data points and the black solid line 104 is a reference line representing the situation where SpHb measurements perfectly match gold standard HgB measurements. The fact that the red regression line 102 has a greater slope than the black solid line 104 indicates that SpHb monitors underestimate the true HgB values for lower HgB levels and overestimate the true HgB values for higher HgB levels. It is important to point out that this phenomenon can also be observed in FIG. 1 of Joseph et al. [10]. However, it appears that this phenomenon has not been specifically reported in the literature that uses SpHb monitors. This implies that the true HgB levels can be considered as a function of SpHb measurements. Below, several linear transformation models are proposed to correct the systematic error observed in FIG. 1.

Let x_{i,1}, x_{i,2}, x_{i,3} . . . x_{i,N}_{ᵢ} denote the SpHb measurements obtained at time t_{i,1}<t_{i,2}<, t_{i,3} . . . <t_{i,N}_{ᵢ}, respectively, for a given gold standard HgB observation i, i=1, 2, . . . 61. For all 61 observations, t_{i,1}=0 and t_{i,N}_{ᵢ}=2, corresponding to the beginning and end of the trailing two hour window right before the gold standard HgB measurement was taken. Due to various constraints, t_{i,1}, t_{i,2}, t_{i,3} . . . t_{i,N}_{ᵢ} might not be equally spaced but the typical frequency of SpHb measurements was 0.5 Hz. Due to variations in measurement frequency and missing observations, the number of SpHb measurements, denoted by Nᵢ associated with different gold standard HgB observation i also varies. Here, t_{i,N}_{ᵢ} is the time instant when the gold standard HgB level is measured. The gold standard HgB level, which is considered as the true level of hemoglobin in medical practice, is denoted as yᵢ.

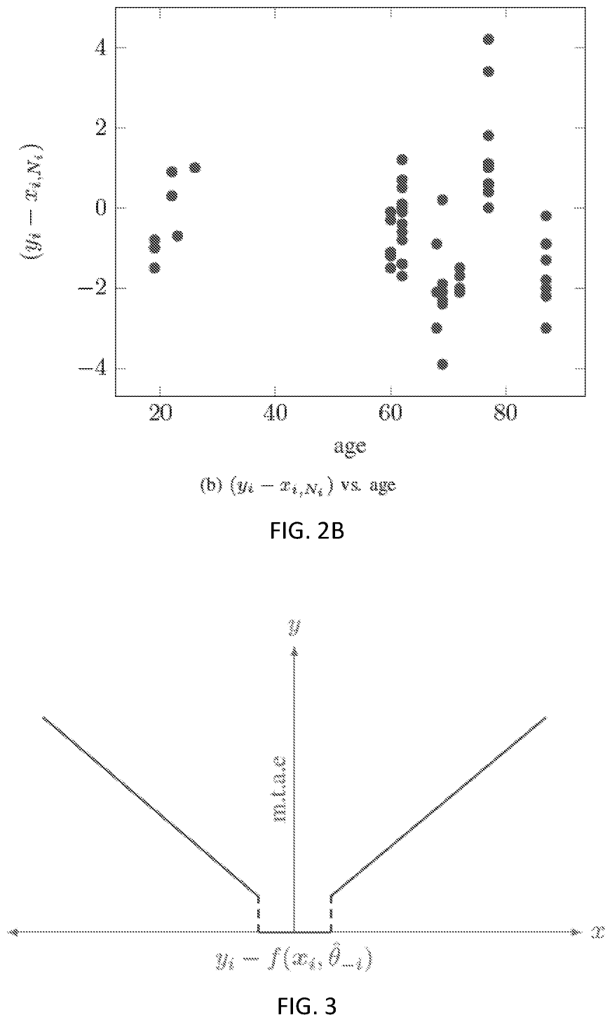

Using above notation, the regression line in FIG. 1 is given as yᵢ=1.275x_{i,N}_{ᵢ}−3.497. The correlation between yᵢ and x_{i,N}_{ᵢ} was 0.80. Note that this value is similar to the correlation for a hemorrhaging patient population reported by Joseph et al. In the dataset, little correlation was found between the SpHb measurement error (i.e., yᵢ−x_{i,N}_{ᵢ}) and patient age or BMI calculated as weight in kilograms divided by square of height in meters. These results are shown in FIGS. 2A-2B. The dataset was not sufficiently large to explore the relationship between the SpHb measurement error and gender or medical complaint because a majority of patients were male and almost all had suffered some sort of trauma.

These analyses indicate the possibility that true HgB levels, which are considered to be given by gold standard laboratory HgB measurements, are a function of SpHb measurements. In the linear models considered here, yᵢ is assumed to be a linear function of the current and prior SpHb measurements given as follows:

$${y}_{i} = {\sum }_{j = 0}^{M} {a}_{j} {x}_{i , {N}_{k} - j} + b . \qquad ( 1 )$$

As described herein, M=0, 19 and 99 are considered. For these three values, the linear model uses the current SpHb measurement, last 20 SpHb measurements, and last 100 SpHb measurements, respectively, before the gold standard HgB level is obtained. These models are given below as:

$$\begin{matrix}{y}_{i} = {a}_{0} {x}_{i , {N}_{i}} + b , & ( I ) \\ {y}_{i} = {\sum }_{j = 0}^{19} {a}_{j} {x}_{i , {N}_{i} - j} + b , \\ and & ( II ) \\ {y}_{i} = {\sum }_{j = 0}^{99} {a}_{j} {x}_{i , {N}_{i} - j} + b . & ( III )\end{matrix}$$

These linear functions can be estimated by minimizing the least square errors, using traditional linear regression estimation methods. To compare the improvement in accuracy of SpHb measurements using these models, a leave one out cross validation approach is used. More specifically, in order to compare the improvement in accuracy for observation i, first the linear transformation parameters â_{i,j} and \hat{b}ᵢ are estimated from all the observations except the ith observation, as follows:

$${\hat{a}}_{i , j} , {\hat{b}}_{i} = {arg min}_{{a}_{j} , b} {\sum }_{k = 1 , k \neq i}^{61} {( {y}_{k} - {\sum }_{j = 0}^{M} {a}_{j} {x}_{k , {N}_{k} - j} + b )}^{2} . \qquad ( 2 )$$

Thus, the predicted value of HgB levels for observation i based on these transformation parameters is given as:

$${\sum }_{j = 0}^{M} {\hat{a}}_{i , j} {x}_{i , {N}_{i} - j} + {\hat{b}}_{i}$$

The absolute prediction error for each gold standard HgB observation i by using the linear transformation approach and raw SpHb measurements are |yᵢ−Σⱼ₌₀^{M}â_{i,j}x_{i,N}_{ᵢ}₋ⱼ\hat{b}ᵢ| and |yᵢ−x_{i,N}_{ᵢ}| respectively. The mean absolute error (m.a.e) is obtained by taking the mean of absolute errors for all observations i=1, 2, . . . 61. The m.a.e values for models (I), (II) and (III) are given in Table II below.

TABLE II
Comparison of prediction accuracy of different linear models
Bold values indicate minimum in the corresponding measure.

| Model | m.a.e | m.t.a.e |
|---|---|---|
| Using x_{i,N}_{ᵢ} to estimate yᵢ | 1.26 g/Dl | 1.21 g/Dl |
| Model (I): yᵢ = a₀x_{i,N}_{ᵢ} + b | 1.13 g/Dl | 1.04 g/Dl |
| Model (II): yᵢ = Σⱼ₌₀¹⁹aⱼx_{i,N}_{ᵢ}₋ⱼ + b | 1.13 g/Dl | 1.06 g/Dl |
| Model (III): yᵢ = Σⱼ₌₀⁹⁹aⱼx_{i,N}_{ᵢ}₋ⱼ + b | 1.17 g/Dl | 1.08 g/Dl |
| Model (IV): yᵢ = β₀ + ∫₀²A(t)xᵢ(t)dt | 1.08 g/Dl | 1.02 g/Dl |
| Model (V): yᵢ = b + a\hat{ξ}_{i,p} | 1.10 g/Dl | 1.02 g/Dl |

Although laboratory based HgB measurements are considered to be the gold standard, studies show that this well-established laboratory-based measurement method is prone to measurement error [11, 12]. According to Gehring et al. [12], an HgB measurement would be clinically acceptable as long as it is within 0.5 g/dl of the true HgB value. Therefore, these new measures of accuracy that account for error in the gold standard HgB value, which is referred to as the mean truncated absolute error (m.t.a.e) and is defined as:

$$m . t . a . e = \frac{1}{61} {\sum }_{i = 1}^{61} \vert {y}_{i} - {\sum }_{j = 0}^{M} {\hat{a}}_{i , j} {x}_{i , {N}_{i} - j} + {\hat{b}}_{i} \vert \times I ( \vert {y}_{i} - {\sum }_{j = 0}^{M} {\hat{a}}_{i , j} {x}_{i , {N}_{i} - j} + {\hat{b}}_{i} \vert \geq 0.5 ) , \qquad ( 3 )$$

where I( ) is the indicator function and is equal to 1 if the expression within the parenthesis is true and 0 otherwise. The intuitive idea behind this new measure is described in FIG. 3 in which the x-axis shows the actual prediction error, and the y-axis shows the value of the prediction error used to quantify the accuracy of the device. Any error less than 0:5 and greater than 0:5 is not considered, because it is assumed that reference HgB value will have some error.

For estimation of parameters in Model (II) and Model (III) a ridge regression penalty is added to the standard least-squares penalty in the estimation problem [13]. The ridge regression penalty ensures that the estimation problem is a strictly convex minimization problem, which may not always be the case when there are missing data and less number of observations. The coefficient of the ridge penalty that gives the minimum leave one out cross validation error from the set of values {10⁻⁵, 10^{-4.5}, . . . 10^{4.5}, 10^{5.0}} was selected. A similar parameter tuning procedure is applied to all penalized regression models discussed herein.

The ridge regression method will now be briefly described using the model (III). The procedure is identical for other least square minimization problems. For the model (III), the minimization problem is to estimate a₀, a₁, . . . a₉₉ is:

$${min}_{{a}_{j} , b} {\sum }_{i} {( {y}_{i} - {\sum }_{j = 0}^{99} {a}_{j} {x}_{i , {N}_{i} - j} + b )}^{2} ,$$

which is not a strictly convex minimization problem as the Hessian matrix is not positive definite. Therefore, a ridge penalty is added to it as follows:

$${min}_{{a}_{j} , b} {\sum }_{i} {( {y}_{i} - {\sum }_{j = 0}^{99} {a}_{j} {x}_{i , {N}_{i} - j} - b )}^{2} + λ ( {\sum }_{j} {a}_{j}^{2} + {b}^{2} ) ,$$

where λ≥0. Different values of λ are tried to obtain a m.a.e by leave one out cross-validation.

As can be seen in Table II, the improvement in prediction accuracy by adding SpHb measurements in Models (I), (II) and (III) indicates that the true HgB levels are indeed a function of prior SpHb measurements. However, comparing m.a.e and m.t.a.e of Model (I) to those of Models (II) and (III) suggests that adding more SpHb observations in the linear transformation approach does not necessarily improve accuracy. There are two possible reasons for this. Firstly, there are missing observations in the SpHb streaming data usually caused by the detachment of the device from the patient for various reasons. Therefore it is important to build an algorithm that is robust against missing data. Secondly, like most physiological signal measuring devices, the SpHb measurements are subject to noise. A regression model similar to Models (II) and (III) does not account for noise in x_{i,j}.

These issues can be addressed by using a functional regression model for predicting true HgB level using streaming SpHb measurements. The key findings from the functional regression model will be discussed first, followed by an attempt to improve the prediction accuracy of the functional regression model through functional principal component analysis.

The first assumption of the functional regression model is that the SpHb measurements x_{i,j} collected over a two-hour window prior to the gold standard Hgb measurement are noisy observations of an underlying smooth function xᵢ(t). As used herein, the smooth function is approximated as:

$${x}_{i} ( t ) = {\sum }_{k = 1}^{K} {∝}_{i , k} {B}_{k} ( t ) , \qquad ( 4 )$$

where B_{K}(t) are a set of B-spline functions.

The definition of a B-spline basis function will now be briefly described. A B-spline function of order n is given as:

$$S ( t ) = {\sum }_{k = 1}^{n} {∝}_{k} {B}_{k , n} ( t ) ,$$

which is defined over tϵ[t₀, tₘ]. Here B_{K,n} is the kth basis function of order n that are recursively defined as follows:

$${B}_{k , 0} = \{ \begin{matrix}1 & {t}_{i} \leq t \leq {t}_{i + 1} \\ 0 & otherwise\end{matrix} \\ {B}_{k , n} ( t ) = \frac{t - {t}_{i}}{{t}_{i + n} - {t}_{n}} {B}_{i , n + 1} ( t ) + \frac{{T}_{i + n + 1} - t}{{T}_{i + n + 1} - t} {B}_{k + 1 , n - 1} ( t )$$

where t₀≤t₁≤ . . . tₘ are the not vectors. Typically they are chosen to be uniformly spaced in the range of spline function [t₀, tₘ]. Additional details are described in [17].

Fourth order splines are used for a uniform set of knot vectors. Here xᵢ(t) is defined over the two hour window tϵ[0, 2]. The number of basis functions, K, is set to 100 for the analysis reported herein. Smoothing using basis functions, such as B-spline functions, achieves two objectives. First, if a considerable number of x_{i,j} are observed, the original signal can be represented using a smaller number of basis functions and coefficients, without losing the in formativeness of the original signal. Secondly, smoothin inherently tends to remove noise from the original signal.

The coefficients ∝_{i,k} are estimated by minimizing:

$${\sum }_{j = 1}^{{N}_{i}} {( {x}_{i} ( {t}_{i , j} ) - {x}_{i , j} )}^{2} + {λ}_{s} {\int }_{0}^{2} {( \frac{{d}^{2} {x}_{i} ( t )}{d {t}^{2}} )}^{2} d t ,$$

Where t_{i,j} represents the time at which SpHb measurement x_{i,j} was taken. Here the term

$${λ}_{s} {\int }_{0}^{2} {( \frac{{d}^{2} {x}_{i} ( t )}{{dt}^{2}} )}^{2}$$

is added to penalize curvature of xᵢ(t) in order to make the estimated functions smooth to avoid overfitting.

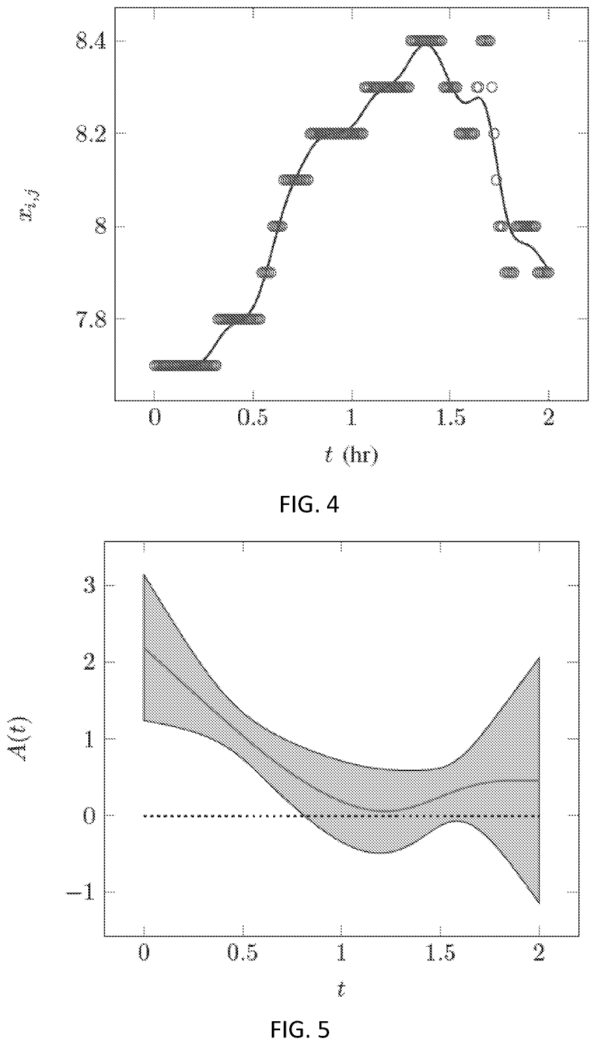

The coefficients that are estimated from (t_{i,j}, x_{i,j}) are denoted by \hat{∝}_{i,k}. The basic functions were B-splines. Although other basis functions can be used, the results were found to be similar. The value λₛ=10⁻² was chosen after comparing cross-validation errors for different magnitudes of smoothing penalties. Details on smoothing using spline functions can be found in Wahba [14] and Ramsay [15, Chapter 4]. The fda-package in R provides several implementation of smoothing and other functional regression algorithms [16]. An example of smoothing SpHb measurements is illustrated in FIG. 4.

Due to different device frequencies used during different measurements and missing data, it may not be always possible that tᵢ_{_{1,j}}=tᵢ_{_{2,j}} for i₁≠i₂, ∀i₁, i₂ϵ{1, 2, . . . 61}. This becomes a challenge to build a regression model like (III). Fitting a smooth function xᵢ(t) to x_{i,j} circumvents these issues. Since xᵢ(t) is estimated by minimizing the least square error, this process can be also interpreted as a way of removing noise from the SpHb measurements.

The functional regression based transformation of SpHb measurements is given as:

yᵢ=β₀+∫₀²A(t)xᵢ(t)dt, (IV)

where A(t) is a smooth coefficient function that establishes the relationship between yᵢ and xᵢ(t), and β₀ is an intercept term. As it is the common practice in functional regression models, the same set of basic functions that were used in (4), are used to define A(t):

$$A ( t ) = {\sum }_{κ = 1}^{K} {β}_{κ} {B}_{κ} ( t ) .$$

The integral in (IV) can be simplified as:

∫₀²A(t)xᵢ(t)dt=αᵢ^{T}Φβ, (5)

where

Φ_{i,j}=∫₀²Bᵢ(t)Bⱼ(t)dt,

and β=[β₁, β₂, . . . , β_{K}]^{T} and αᵢ=[α_{i,1}, α_{i,2}, . . . , α_{i,k}]^{T}. Further, by defining X=[1αᵢ^{T}Φ], where 1 is a vector with all elements equal to 1, the transformation in (IV) is given as

$$y = X [ \begin{matrix}{β}_{0} \\ β\end{matrix} ] .$$

Linear regression models such as Model I, II, and II use coefficients to approximate HgB as linear combination of past SpHb values as Σⱼaⱼx_{i,N}_{ᵢ}₋ⱼ+b. Likewise, the functional regression model approximates HgB values as ∫A(t)xᵢ(t)+β₀ based on the smoothed SpHb signal xᵢ(t). Since xᵢ(t) is linear combination of basis functions, the functional regression model can be viewed as a linear model in the space of basic functions. Typically, a less than N, number of basis function is necessary to fit splines to the x_{i,j}. Thus the calculation of the functional regression model could be less computationally expensive than using SpHb measurements directly. This makes the predictive model implementable on devices that have embedded processors with lesser computational power, like the SpHb monitors.

As was done in the case of linear transformations, a functional regression based transformation of x_{i,j} is given as:

∫₀²Â(t)xᵢ(t)dt,

where Âᵢ(t)=\hat{β}₀Σ_{K=1}^{K}\hat{β}_{K,i}B_{K}(t) is estimated by the following minimization:

$$[ {\hat{β}}_{0 , i} , {\hat{β}}_{i} ] = {arg min}_{{β}_{0} , β} {\sum }_{k = 1 , k \neq i}^{61} {( {y}_{k} - {β}_{0} - {\int }_{0}^{2} A ( t ) {x}_{k} ( t ) dt )}^{2} + {λ}_{r} {\int }_{0}^{2} {[ \frac{{d}^{2} {A}_{i} ( t )}{{dt}^{2}} ]}^{2} dt$$

Again, the term

$${λ}_{r} {\int }_{0}^{2} {[ \frac{{d}^{2} {A}_{i} ( t )}{{dt}^{2}} ]}^{2} d t$$

is added to ensure A(t) is smooth and prevents over-fitting. The penalty term A, was chosen from a set of values {10⁻⁵, 10^{-4.5}, . . . 10^{4.5}, 10^{5.0}}. The value that minimized m.a.e. for leave one out cross validation was selected.

The leave one out cross-validation m.a.e, and m.t.a.e for all regression models are reported in Table II. The m.a.e, and m.t.a.e for functional regression model (IV) was the lowest, providing evidence that smoothing the noisy SpHb observations improved the accuracy. The coefficient function was further analyzed to understand the relationship between the SpHb and the true Hgb levels. FIG. 5 shows the coefficient function as a solid line and the confidence interval around it as gray shaded area. The coefficient function can be interpreted similarly to the coefficients from regular linear regression models. If the confidence interval of A(t) for some t contains zero, it can be interpreted as A(t) not being significant for that time t. Hence, based on this data set, from FIG. 5, SpHb observation obtained 30 to 45 minutes prior are not significant predictors of current HgB levels. Larger data sets could be used to more definitively determine the length of the prior time window for developing such methods.

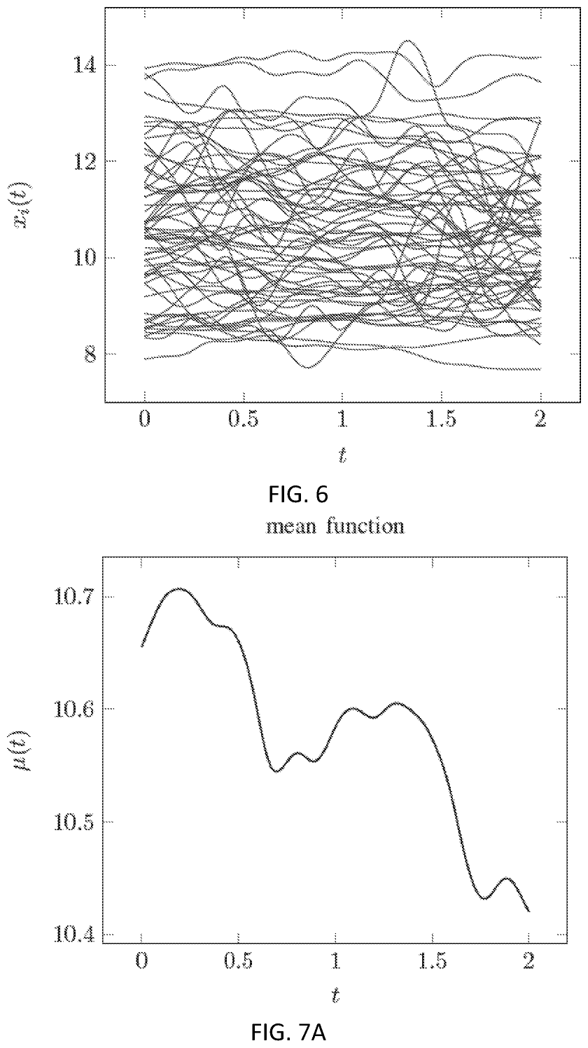

In FIG. 6, the smoothed SpHb measurements from all 61 patients are shown. It is hard to assess the variability of SpHb measurements in the dataset from FIG. 6. Therefore, a functional principal component analysis (FPCA) is performed on the smoothed SpHb measurements [16]. The basic idea of FPCA is to represent each xᵢ(t) as a linear combination of mean functions and a set of principal components:

$${x}_{i} ( t ) = μ ( t ) + {\sum }_{p} {\hat{ξ}}_{i , p} {ψ}_{p} ( t ) . \qquad ( 6 )$$

Here, μ(t) is the mean function of all xᵢ(t)s. The functions ψₚ(t) are the functional principal components of xᵢ(t), which explain the variability in xᵢ(t). The \hat{ξ}_{i,p} are principal component scores that quantify the variability of xᵢ(t) along ψₚ(t). The mean of each \hat{ξ}_{i,p} is zero for all p and the standard deviation of \hat{ξ}_{i,p} for each p indicates the amount of variation in xᵢ(t) that is explained by ψₚ(t).

If each xᵢ(t) is considered a random function, μ(t) is the mean of all such functions, then the function xᵢ(t)−μ(t) and be viewed as random perturbations with mean equal to zero. In FPCA, these random perturbations, given by the variance V(t) of xᵢ(t) at time t, can be split into components:

$$V ( t ) = {\sum }_{p = 1}^{∞} {ξ}_{p} {ψ}_{p} ( t ) ,$$

where ξₚ are normally distributed random variables. The variance of ξₚ represents the amount of variation in xᵢ(t) that can be attributed to ψₚ(t).

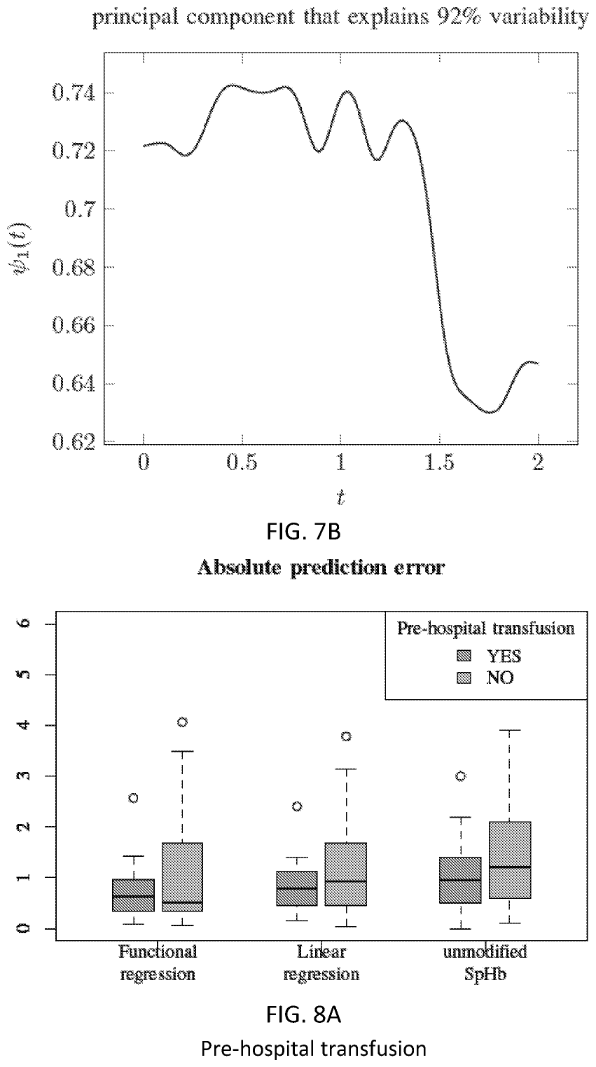

In FIGS. 7A-7B, the mean function of xᵢ(t)s and the principal component that explains 92% of the variability is shown. As can be seen from the leading functional principal component, most of the variability in SpHb measurement of the current dataset occur 0.5 hours prior to the gold standard HgB measurement. Therefore, A(t) for t≤0.5 was found to be more significant. Let \hat{ξ}_{i,1} be the principal component scores for the leading principal component. The following model:

yᵢ=b+a\hat{ξ}_{i,p}, (V)

can be used to predict HgB values using only the principal component scores. The m.a.e. for model (V) was found to be 1.10 g/Dl, which is comparable with model (IV) (See Table II for m.a.e., and m.t.a.e values). This result further proves that the smoothing of SpHb measurements leads to better accuracy in predicting true HgB levels.

The previously described models were developed using data from multiple patients. The performance of these models will now be analyzed when only data from an individual patient is used. the patients with ID's 3, 5, 6, and 12, who have more than five true HgB measurements, will be discussed.

In Table III the m.a.e of leave one out cross-validation for a few selected models built using individual patient data is compared. Although there is some improvement in prediction accuracy by using functional regression compared to raw SphB measurements, the amount of improvement varies from patient to patient. Patient 12 has 12 observations and it has the highest improvement in prediction accuracy. This indicates that prediction accuracy of SpHb monitors can be improved by calibrating the prediction model for an individual patient.

TABLE III
Mean absolute error of different models for different patients

| | Sample | Using x_{i,N}_{ᵢ} to | | | |
|---|---|---|---|---|---|
| Patient | size | estimate yᵢ | Model (I) | Model (II) | Model (V) |
| 3 | 7 | 1.33 | 0.51 | 0.53 | 0.58 |
| 5 | 6 | 0.47 | 0.81 | 0.54 | 0.67 |
| 6 | 6 | 2.13 | 0.36 | 1.62 | 0.51 |
| 12 | 12 | 1.27 | 1.06 | 1.02 | 1.01 |

Additional analysis was also performed by taking only one (the first) true HgB observation from each patient. Thus the data set consisted of 14 observations. These results are presented in Table IV. These results further show that the functional regression model does show improvement in prediction compared to linear models. It further validates the claim that smoothing the SpHb measurements improves SpHb prediction accuracy.

TABLE IV
Comparison of prediction accuracy of different
linear models (one observation per patient)

| Model | m.a.e |
|---|---|
| Using x_{i,N}_{ᵢ} to estimate yᵢ | 1.35 g/Dl |
| Model (I): yᵢ = a₀x_{i,N}_{ᵢ} + b | 1.22 g/Dl |
| Model (II): yᵢ = Σⱼ₌₀¹⁹aⱼx_{i,N}_{ᵢ}₋ⱼ + b | 1.55 g/Dl |
| Model (III): yᵢ = Σⱼ₌₀⁹⁹aⱼx_{i,N}_{ᵢ}₋ⱼ + b | 1.72 g/Dl |
| Model (IV): yᵢ = β₀ + ∫₀²A(t)xᵢ(t)dt | 1.14 g/Dl |
| Model (V): yᵢ = b + a\hat{ξ}_{i,p} | 1.06 g/Dl |

The impact of various clinical factors on the prediction accuracy of the proposed models will now be discussed. The clinical factors considered are:

1) Blood transfusion provided to patients before they arrived to hospital.

2) Blood transfusion provided in the hospital.

3) If the patient used any anticoagulant or had any bleeding disorder.

4) The type of trauma blunt, penetrating or crushing.

Table V summarizes the different clinical factors considered and the number of data points (observations) and number of patients for each category. Here, an observation refers to a laboratory measurement of HgB levels with a trailing window of SpHb measurements. Since some patients have more than one laboratory-based HgB measurement, there are more observations than patients.

TABLE V
Number of patients and observations
for different clinical factors

| Did the patient receive blood | Number of | Number of |
|---|---|---|
| before arrival to hospital? | Observations | Patients |
| No | 22 | 6 |
| Yes | 22 | 6 |
| No information | 17 | 2 |
| Did the patient receive | Number of | Number of |
| blood in hospital? | Observations | Patients |
| No | 5 | 3 |
| Yes | 56 | 11 |
| Anticoagulant or | Number of | Number of |
| bleeding disorder? | Observations | Patients |
| No | 39 | 10 |
| Yes | 22 | 4 |
| | Number of | Number of |
| Type of trauma? | Observations | Patients |
| Blunt | 21 | 6 |
| Crush | 5 | 2 |
| Penetrating | 5 | 1 |
| Other | 30 | 5 |

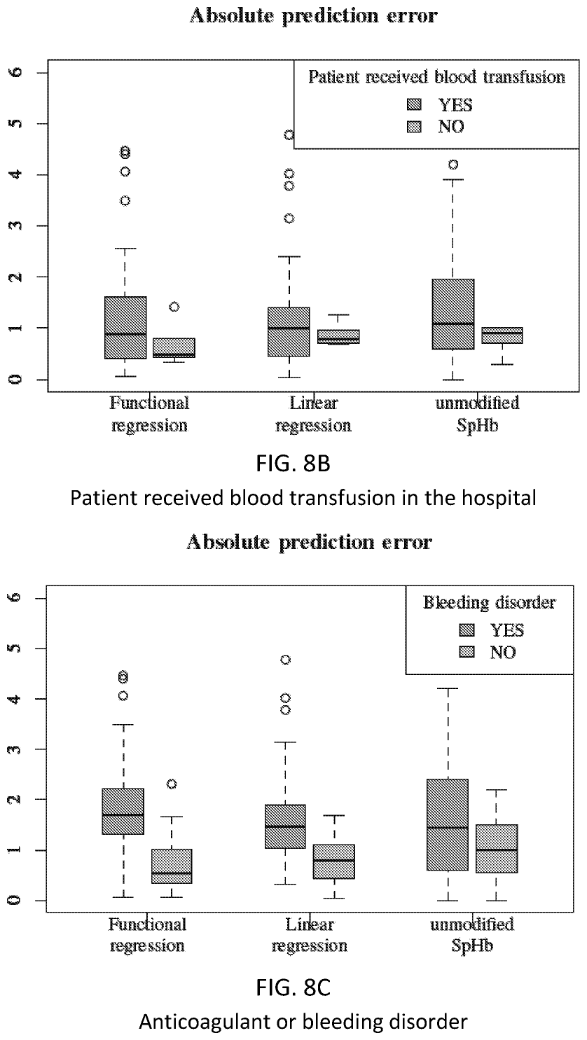

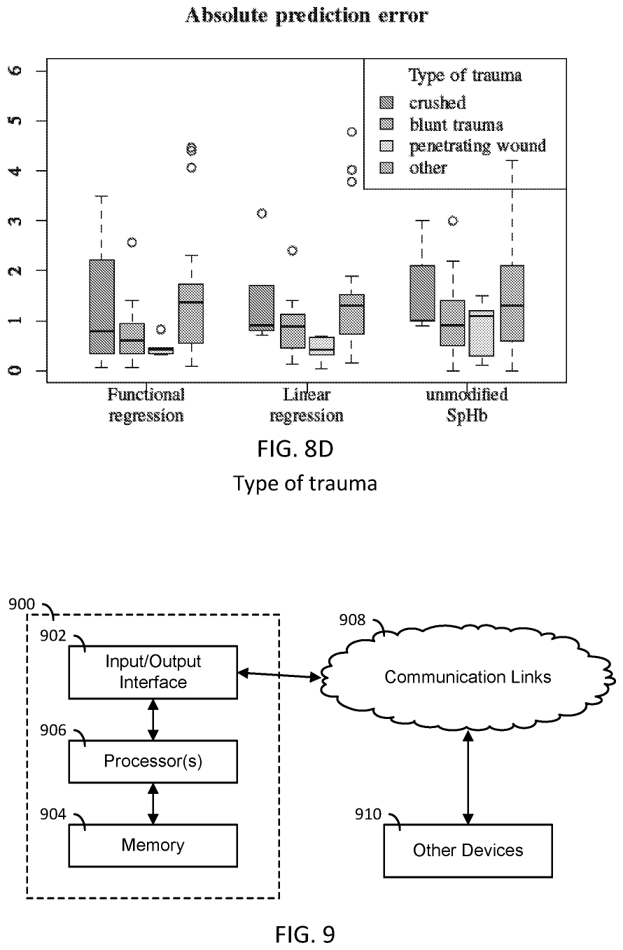

FIGS. 8A-8D show the absolute value of prediction error for the three different algorithms. As before, the leave-one-out cross-validation approach is used to calculate the prediction error. Based on FIGS. 8A and 8B, there is not sufficient evidence to conclude that blood transfusion has an impact on the accuracy of the SpHb monitors. Similarly, FIG. 8D shows that the type of trauma also does not have a significant effect on the device accuracy. However, use of anticoagulant or presence of bleeding disorders seem to have a negative impact on SpHb monitor accuracy, as shown in FIG. 8C.

The analyses presented here and in some prior research show that the accuracy of SpHb depends on the level of HgB levels. Thus, past SpHb measurements can be used to improve the accuracy of SpHb monitors. Also, like most physiological signals, SpHb monitors have noisy outputs. The functional regression model smooths and thus removes some of the noise from the data. This was shown to improve prediction accuracy. Extensions like functional principal component analysis also showed that fitting a smooth function to SpHb measurement data did improve the accuracy of Hgb predictions.

The foregoing analysis shows that there is variability in SpHb based prediction from patient to patient. FIGS. 2A-2B also shows that age and BMI might be related to the variability in accuracy. Effect of factors such as skin color and use of anticoagulant should be considered as well.

The models described herein can predict future SpHb values. Such predictions are critical to determine the optimal timing of transfusion and thus help plan the amount of blood product needed in situations where blood products are scarce. Instead of a data-driven prediction algorithm, models for hemoglobin dynamics could also be developed using SpHb measurements. These realistic dynamical models can lead to individualized stochastic filtering algorithms to guide blood transfusion decisions.

Now referring to FIG. 9, an apparatus 900 for determining a current hemoglobin level in accordance with the present invention is shown. The apparatus 900 can be a server computer, a workstation computer, a laptop computer, a mobile communications device, a personal data assistant, a medical device or any other device capable of performing the functions described herein. The apparatus 900 includes an input/output interface 902, a memory 904, and one or more processors 906 communicably coupled to the input/output interface 902 and the memory 904. Note that the apparatus 900 may include other components not specifically described herein. The memory 904 can be local, remote or distributed. Likewise, the one or more processors 906 can be local, remote or distributed. The input/output interface 902 can be any mechanism for facilitating the input and/or output of information (e.g., web-based interface, touchscreen, keyboard, mouse, display, printer, etc.) Moreover, the input/output interface 902 can be a remote device communicably coupled to the one or more processors 906 via one or more communication links 908 (e.g., network(s), cable(s), wireless, satellite, etc.). The one or more communication links 908 can communicably couple the apparatus 900 to other devices 910 (e.g., databases, remote devices, hospitals, doctors, researchers, patients, etc.).

The one or more processors 906 receive a series of hemoglobin measurements taken within a time window, determine a current hemoglobin level based on the series of hemoglobin measurements using a linear regression model, and provide the current hemoglobin level via the input/output interface 902. In one aspect, the one or more processors 906 receive a selection of the time window based on a specified number of hemoglobin measurements via the input/output interface 902. In another aspect, the one or more processors 906 determine and provide the current hemoglobin level only after the time window is complete. In another aspect, the time window is a rolling time window and the one or more processors 906 determine and provide the current hemoglobin level for each new hemoglobin measurement. In another aspect, the one or more processors 906 receive a selection of the linear regression model from a group of at least two models comprising: yᵢ=a₀x_{i,N}_{ᵢ}+b where yᵢ is the current hemoglobin level, a is a first coefficient, x_{i,N}_{ᵢ} is the hemoglobin measurement, and b is a second coefficient; yᵢ=Σⱼ₌₀^{M}aⱼx_{i,N}_{ᵢ}₋ⱼ+b where M is a number of hemoglobin measurements taken within the time window; yᵢ=β₀+∫₀²A(t)xᵢ(t)dt where β₀ is an intercept term, xᵢ(t) is a smoothing function, and A(t) is a smooth coefficient function that establishes a relationship between yᵢ and xᵢ(t); or yᵢ=b+a\hat{ξ}_{i,p} where \hat{ξ}_{i,p} are principal component scores that quantify the variability of xᵢ(t) along ψₚ(t), and ψₚ(t) are functional principal components of xᵢ(t) explaining the variability in xᵢ(t). In another aspect, the one or more processors 906 receive or determine the first coefficient and the second coefficient. In another aspect, the one or more processors 906 smooth the series of hemoglobin measurements using a B-spline basis function. In another aspect, the series of hemoglobin measurements are received from the input/output interface 902, or one or more sensors communicably coupled to the one or more processors 906, or the memory 904. In another aspect, a time interval between the hemoglobin measurements is not equally spaced. In another aspect, the series of hemoglobin measurements includes missing data or outlier data. In another aspect, the one or more processors 906 adjust the current hemoglobin level based on one or more prior laboratory determined hemoglobin levels. In another aspect, the one or more processors 906 send an alert via the input/output interface whenever the current hemoglobin level or a rate of change of the current hemoglobin level is outside one or more limits. In another aspect, the one or more processors 906 receive one or more patient factors via the input/output interface 902, one or more sensors or one or more modules. In another aspect, the one or more patient factors comprise a heart rate, an arterial oxygen saturation, an intravascular volume, a Pleth variability index, a perfusion index, a total oxygen content, an age, an injury type, a Glasgow coma index, a sex, a body mass index, a systolic blood pressure, or a diastolic blood pressure. In another aspect, the one or more processors 906 adjust the current hemoglobin level based on the one or more patient factors. In another aspect, the one or more sensors or the one or more modules comprise a pulse oximeter, an intravascular volume estimator, or a patient medical data source. In another aspect, the one or more processors 906: determine a blood product or fluid amount, a blood product or fluid type, a transfusion rate, or a transfusion timing based on the current hemoglobin level and the one or more patient factors; and provide the blood product or fluid amount, the blood product of fluid type, the transfusion rate, or the transfusion timing via the input/output interface 902. In another aspect, the blood product or fluid amount of the blood product or fluid type are administered to a patient at the transfusion rate and the transfusion timing using one or more devices communicably coupled to the input/output interface.

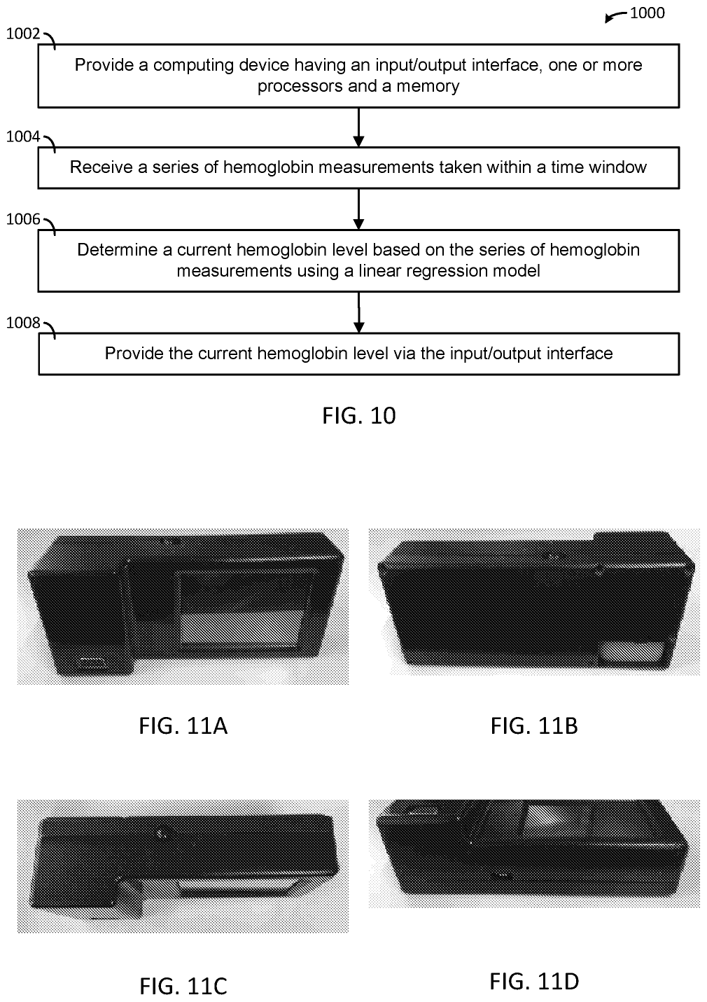

Referring now to FIG. 10, a flow chart of a computerized method 1000 of determining a current hemoglobin level is shown. A computing device having an input/output interface, one or more processors and a memory is provided in block 1002. A series of hemoglobin measurements taken within a time window are received in block 1004. A current hemoglobin level is determined based on the series of hemoglobin measurements using a linear regression model in block 1006. The current hemoglobin level is provided via the input/output interface in block 1008.

In one aspect, the method further comprises selecting the time window based on a specified number of hemoglobin measurements. In another aspect, the determining and providing steps are performed only after the time window is complete. In another aspect, the time window is a rolling time window and the determining and providing steps are repeated for each new hemoglobin measurement. In another aspect, the method further comprises selecting the linear regression model from a group of at least two models comprises: yᵢ=a₀x_{i,N}_{ᵢ}+b where yᵢ is the current hemoglobin level, a is a first coefficient, x_{i,N}_{ᵢ} is the hemoglobin measurement, and b is a second coefficient; yᵢ=Σⱼ₌₀^{M}aⱼx_{i,N}_{ᵢ}₋ⱼ+b where M is a number of hemoglobin measurements taken within the time window; yᵢ=β₀+∫₀²A(t)xᵢ(t)dt where β₀ is an intercept term, xᵢ(t) is a smoothing function, and A(t) is a smooth coefficient function that establishes a relationship between yᵢ and xᵢ(t); or yᵢ=b+a\hat{ξ}_{i,p} where \hat{ξ}_{i,p} are principal component scores that quantify the variability of xᵢ(t) along ψₚ(t), and ψₚ(t) are functional principal components of xᵢ(t) explaining the variability in xᵢ(t). In another aspect, the method further comprises receiving or determining the first coefficient and the second coefficient. In another aspect, the method further comprises smoothing the series of hemoglobin measurements using a B-spline basis function. In another aspect, the series of hemoglobin measurements are received from the input/output interface, or one or more sensors communicably coupled to the one or more processors, or the memory. In another aspect, a time interval between the hemoglobin measurements is not equally spaced. In another aspect, the series of hemoglobin measurements includes missing data or outlier data. In another aspect, the method further comprises adjusting the current hemoglobin level based on one or more prior laboratory determined hemoglobin levels. In another aspect, the method further comprises sending an alert via the input/output interface whenever the current hemoglobin level or a rate of change of the current hemoglobin level is outside one or more limits. In another aspect, the method further comprises receiving one or more patient factors via the input/output interface, one or more sensors or one or more modules. In another aspect, the one or more patient factors comprise a heart rate, an arterial oxygen saturation, an intravascular volume, a Pleth variability index, a perfusion index, a total oxygen content, an age, an injury type, a Glasgow coma index, a sex, a body mass index, a systolic blood pressure, a diastolic blood pressure, or a transfusion target range. In another aspect, the method further comprises adjusting the current hemoglobin level based on the one or more patient factors. In another aspect, the one or more sensors or the one or more modules comprise a pulse oximeter, an intravascular volume estimator, or a patient medical data source. In another aspect, the method further comprises: determining a blood product of fluid amount, a blood product of fluid type, a transfusion rate, or a transfusion timing based on the current hemoglobin level and the one or more patient factors; and providing the blood product or fluid amount, the blood product or fluid type, the transfusion rate, or the transfusion timing via the input/output interface. In another aspect, the method further comprises administering the blood product or fluid amount of blood product or fluid type to a patient at the transfusion rate and transfusion timing using one or more devices communicably coupled to the input/output interface. In another aspect, the input/output interface comprises a remote device, and the remote device is communicably coupled to the one or more processors via one or more networks. In another aspect, the computing device comprises a server computer, a workstation computer, a laptop computer, a mobile communications device, a personal data assistant, or a medical device.

Moreover, the method can be implemented using a non-transitory computer readable medium that when executed causes the one or more processors to perform the method.

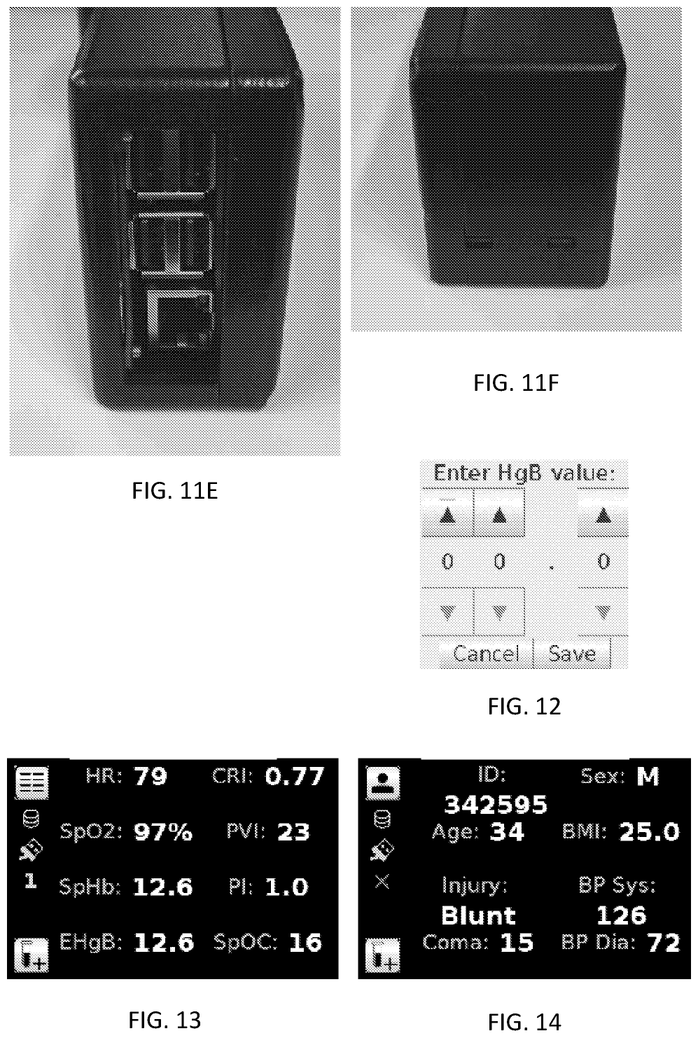

Now referring to FIGS. 11A-11F and 12-14, an apparatus in accordance with another embodiment of the present invention is shown. The Heme device is a data-collecting appliance which serves two purposes. First, it allows practitioners to easily monitor changes in a patient's vital signs. And second, it can be used by medical researchers to improve the accuracy of hemoglobin estimation algorithms. It gathers and stores data from a Masimo Radical7 and a Flashback Technologies CipherOx and will compute an estimated HgB value from the observed SpHb.

FIG. 11A is an image showing the front of the device with the touchscreen and the connector for the CipherOx's finger clip. FIG. 11B is an image showing the back of the device with the slot where the CipherOx device is inserted as well as the Micro USB connector used to charge the CipherOx device. FIG. 11C is an image showing the top of the device with the power switch. FIG. 11D is an image showing the bottom of the device with the Micro USB port used to charge the device. FIG. 11E is an image showing the right side of the device with the USB and Ethernet ports. The USB ports are used to connect the Radical7 device, charge the CipherOx device and data storage devices. FIG. 11F is an image of the left side of the device two holes that are used to press the buttons on the CipherOx device without removing it from the Heme device. The device contains a 2500 mAh Lithium Ion battery. It is recommended to maintain power by the Micro USB port on the bottom under normal usage.

The device's graphical user interface consists of three screens. The screens can be changed by pushing the button in the upper-left corner of the screen. Each page also contains three status icons describing the internal status of the system. The storage status icon takes the form of a stack of disks. If the storage is unavailable (either due to missing USB drive, or the storage device is still being mounted), the icon will show red and have an “X” across it. When the storage is available and the device able to save data, the icon will show green. The external device status icon takes the form of a USB plug. If the Heme device was not able to detect the Radical7 device, the icon will show red and have an “X” across it. If the Radical7 device was found and successfully connected, the icon will show green. The estimate status icon takes the form of a red “X” or different green numbers. This icon shows the status of the HgB estimate models. If the model is not being used (likely due to lack of SpHb data), the icon will be a red “X”. If an estimate was able to be generated, the icon will represent which model was used to generate the estimate. Examples include N=1, 20 or 100 for the linear regression model.

FIG. 12 is an image of a HGB picker widget screen. A button in the lower-left of the screen allows for reporting of ground-truth HgB values (e.g., from patient lab work). This value is added to the data files to make it easier to correlate true HgB values to estimates.

FIG. 13 is an image of a details page screen. The details page shows eight data points, gathered by various parts of the system. The points are gathered by the Radical7 device. The CRI data point is from the CipherOx device. The EHgB is the estimated HgB value calculated on the Heme device itself using one of more of the models described above. The data points are as follows:

| Data Point | Meaning | Source |
|---|---|---|
| HR | Heart rate | Radical7 Device |
| SpO2 | Arterial oxygen saturation | Radical7 Device |
| SpHb | Hemoglobin | Radical7 Device |
| | (observed from Radical7) | |
| EHgB | Estimated hemoglobin | Heme Device |
| CRI | Compensatory reserve index | CipherOx Device |
| PVI | Pleth variability index | Radical7 Device |
| PI | Perfusion index | Radical7 Device |
| SpOC | Total oxygen content | Radical7 Device |

FIG. 14 is an image of a patient page screen. The patient page shows metadata about the patient. A patient ID is generated by the Heme system and used in naming the data files and differentiating between runs of the system. The meta data is as follows:

| Data Point | Meaning | Values |
|---|---|---|
| ID | Internally generated identifier | Random 6-digit number |
| Age | | 0-199 |
| Injury | | Blunt, Penetrating, |
| | | Non-Trauma, or N/A |
| Coma | Glasgow Coma Scale | 3-15 |
| Sex | | Female, Male, or N/A |
| BMI | Body mass index | 0.0-50.0 |
| BP Sys | Systolic blood pressure | 0-199 |
| BP Dia | Diastolic blood pressure | 0-199 |

The device inputs include Masimo Radical7, Flashback Technologies CipherOx, and data storage. The Masimo Radical7 is a pulse oximeter measuring several different data points. The Heme device opens a serial connection to the Radical7 and retrieves the data once per second. The Flashback Technologies CipherOx is a device using pulse oximetry to calculate its proprietary Compensatory Reserve Index (CRI). The CRI tries to measure intravascular volume, relative to the individual patient's response to hypovolemia. Data is stored in an encrypted container file on a prepared USB drive. The container file is created with VeraCrypt (https://veracrypt.fr), which keeps the contents of the container encrypted. When the Heme system launches, it mounts the encrypted container and writes the data files there. The data is saved in CSV format in five files:

| Filename | Description |
|---|---|
| <id>_cri.csv | Data from the CipherOx device |
| <id>_hgb.csv | Estimated and ground-truth HgB values |
| <id>_patient.csv | Patient metadata |
| <id>_vitals.csv | Data from the Radical7 device |
| <id>_master.csv | A combination of the above four |

The master document will write a new entry when any new data is available. This may result in multiple repeated lines, but is a tradeoff for having a single file. If there is no prepared USB drive is inserted into the Heme device, no data will be saved.

The Heme system calculates estimated hemoglobin levels using a linear regression model. It allows for three models of differing values for N. The default values are N=1, N=20, and N=100. The model files are configured by a specially formatted JSON file that encodes the α and β coefficients. An example model coefficient file for N=1 is shown here:

```
{
  “alpha”: [
    1.275
  ],
  “beta”: −3.497
}
```

When constructing the linear regression files, the value for N is determined by the length of the “alpha” array in the coefficient file. If there are files named “model1.json”, “model2.json”, and “model3.json” on the HEMESTORAGE USB drive, the Heme system will copy those internally and use them for future estimation. The N values must be increasing in size (i.e. the N value for model 1 must be smaller than the N value for model 2, etc. . . . ).

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

[1] G. Bourner, J. Dhaliwal, and J. Sumner, “Performance evaluation of the latest fully automated hematology an-alyzers in a large, commercial laboratory setting: a 4-way, side-by-side study.” Laboratory hematology: official publication of the International Society for Laboratory Hematology, vol. 11, no. 4, pp. 285-297, 2005.
[2] J. B. Holcomb, N. R. McMullin, L. Pearse, J. Caruso, E. Wade, L., Oetjen-Gerdes, H. R. Champion, M. Lawnick, W. Farr, S. Rodriguez et al., “Causes of death in us special operations forces in the global war on terrorism: 2001-2004,” Annals of surgery, vol. 245, no. 6, pp. 986-991, 2007.
[3] B. Mitra, M. Fitzgerald, and J. Chan, “The utility of a shock index 1 as an indication for pre-hospital oxygen carrier administration in major trauma,” Injury, vol. 45, no. 1, pp. 61-65, 2014.
[4] M. D. Zielinski, G. A. Wilson, P. M. Johnson, S. F. Polites, D. H. Jenkins, W. S. Harmsen, J. B. Hol-comb, C. E. Wade, D. J. Del Junco, E. E. Fox et al., “Ideal hemoglobin transfusion target for resuscitation of massive-transfusion patients,” Surgery, vol. 160, no. 6, pp. 1560-1567, 2016.
[5] D. Frasca, C. Dahyot-Fizelier, K. Catherine, Q. Levrat, B. Debaene, and O. Mimoz, “Accuracy of a continuous noninvasive hemoglobin monitor in intensive care unit patients,” Critical care medicine, vol. 39, no. 10, pp. 2277-2282, 2011.
[6] B. Joseph, P. Hadjizacharia, H. Aziz, K. Snyder, J. Wynne, N. Kulvatunyou, A. Tang, T. OKeeffe, R. Latifi, R. Friese et al., “Continuous noninvasive hemoglobin monitor from pulse ox: ready for prime time?” World journal of surgery, vol. 37, no. 3, pp. 525-529, 2013.
[7] M. Patino, L. Schultz, M. Hossain, J. Moeller, M. Mah-moud, J. Gunter, and C. D. Kurth, “Trending and accu-racy of noninvasive hemoglobin monitoring in pediatric perioperative patients,” Anesthesia & Analgesia, vol. 119, no. 4, pp. 920-925, 2014.
[8] F. Yao, H.-G. Muller, and J.-L. Wang, “Functional data analysis for sparse longitudinal data,” Journal of the American Statistical Association, vol. 100, no. 470, pp. 577-590, 2005.
[9] M. J. Daniels and J. W. Hogan, Missing data in lon-gitudinal studies: Strategies for Bayesian modeling and sensitivity analysis. CRC Press, 2008.
[10] B. Joseph, V. Pandit, H. Aziz, N. Kulvatunyou, B. Zang-bar, A. Tang, T. OKeeffe, Q. Jehangir, K. Snyder, and P. Rhee, “Transforming hemoglobin measurement in trauma patients: noninvasive spot check hemoglobin,” Journal of the American College of Surgeons, vol. 220, no. 1, pp. 93-98, 2015.
[11] K. P. Patel, G. W. Hay, M. K. Cheteri, and D. W. Holt, “Hemoglobin test result variability and cost analysis of eight different analyzers during open heart surgery,” The Journal of extra-corporeal technology, vol. 39, no. 1, p. 10, 2007.
[12] H. Gehring, C. Hornberger, L. Dibbelt, A. Roth-Isigkeit, K. Gerlach, J. Schumacher, and P. Schmucker, “Accuracy of point-of-care-testing (poct) for determining hemoglobin concentrations,” Acta Anaesthesiologica Scandinavica, vol. 46, no. 8, pp. 980-986, 2002.
[13] A. E. Hoerl and R. W. Kennard, “Ridge regression: Biased estimation for nonorthogonal problems,” Techno-metrics, vol. 12, no. 1, pp. 55-67, 1970.
[14] G. Wahba, “Smoothing noisy data with spline functions,” Numerische Mathematik, vol. 24, no. 5, pp. 383-393, 1975.
[15] J. O. Ramsay, Functional data analysis. Wiley Online Library, 2006.
[16] J. Ramsay, H. Wickham, S. Graves, and G. Hooker, “fda: Functional data analysis. r package version 2.2. 6,” 2010.
[17] M. Unser, A. Aldroubi, and M. Eden, “B-spline signal processing. i. theory,” IEEE transactions on signal pro-cessing, vol. 41, no. 2, pp. 821-833, 1993.

1. A computerized method of determining a current hemoglobin level comprising:
providing a computing device having an input/output interface, one or more processors and a memory;
receiving a series of hemoglobin measurements taken within a time window;
determining a current hemoglobin level based on the series of hemoglobin measurements using a linear regression model; and
providing the current hemoglobin level via the input/output interface.

2. The method of claim 1, further comprising selecting the time window based on a specified number of hemoglobin measurements.

3. The method of claim 1, wherein the determining and providing steps are performed only after the time window is complete.

4. The method of claim 3, wherein the time window is a rolling time window and the determining and providing steps are repeated for each new hemoglobin measurement.

5. The method of claim 1, further comprising selecting the linear regression model from a group of at least two models comprising:
yᵢ=a₀x_{i,N}_{ᵢ}+b where yᵢ is the current hemoglobin level, a is a first coefficient, x_{i,N}_{ᵢ} is the hemoglobin measurement, and b is a second coefficient;
yᵢ=Σⱼ₌₀^{M}aⱼx_{i,N}_{ᵢ}₋ⱼ+b where M is a number of hemoglobin measurements taken within the time window;
yᵢ=β₀+∫₀²A(t)xᵢ(t)dt where β₀ is an intercept term, xᵢ(t) is a smoothing function, and A(t) is a smooth coefficient function that establishes a relationship between yᵢ and xᵢ(t); or
yᵢ=b+a\hat{ξ}_{i,p} where \hat{ξ}_{i,p} are principal component scores that quantify the variability of xᵢ(t) along ψₚ(t), and ψₚ(t) are functional principal components of xᵢ(t) explaining the variability in xᵢ(t).

6. The method of claim 5, further comprising receiving or determining the first coefficient and the second coefficient.

7. The method of claim 1, further comprising smoothing the series of hemoglobin measurements using a B-spline basis function.

8. The method of claim 1, wherein the series of hemoglobin measurements are received from the input/output interface, or one or more sensors communicably coupled to the one or more processors, or the memory.

9. The method of claim 1, wherein a time interval between the hemoglobin measurements is not equally spaced.

10. The method of claim 1, wherein the series of hemoglobin measurements includes missing data or outlier data.

11. The method of claim 1, further comprising adjusting the current hemoglobin level based on one or more prior laboratory determined hemoglobin levels.

12. The method of claim 1, further comprising sending an alert via the input/output interface whenever the current hemoglobin level or a rate of change of the current hemoglobin level is outside one or more limits.

13. The method of claim 1, further comprising receiving one or more patient factors via the input/output interface, one or more sensors or one or more modules.

14. The method of claim 13, wherein the one or more patient factors comprise a heart rate, an arterial oxygen saturation, an intravascular volume, a Pleth variability index, a perfusion index, a total oxygen content, an age, an injury type, a Glasgow coma index, a sex, a body mass index, a systolic blood pressure, a diastolic blood pressure, or a transfusion target range.

15. The method of claim 13, further comprising adjusting the current hemoglobin level based on the one or more patient factors.

16. The method of claim 13, wherein the one or more sensors or the one or more modules comprise a pulse oximeter, an intravascular volume estimator, or a patient medical data source.

17. The method of claim 13, further comprising:
determining a blood product of fluid amount, a blood product of fluid type, a transfusion rate, or a transfusion timing based on the current hemoglobin level and the one or more patient factors; and
providing the blood product or fluid amount, the blood product or fluid type, the transfusion rate, or the transfusion timing via the input/output interface.

18. The method of claim 17, further comprising administering the blood product or fluid amount of blood product or fluid type to a patient at the transfusion rate and transfusion timing using one or more devices communicably coupled to the input/output interface.

19. The method of claim 1, wherein the input/output interface comprises a remote device, and the remote device is communicably coupled to the one or more processors via one or more networks.

20. (canceled)

21. An apparatus for determining a current hemoglobin level comprising:
an input/output interface;
a memory; and
one or more processors communicably coupled to the input/output interface and the memory, wherein the one or more processors receive a series of hemoglobin measurements taken within a time window, determine a current hemoglobin level based on the series of hemoglobin measurements using a linear regression model, and provide the current hemoglobin level via the input/output interface.

22-40. (canceled)
